(19) United States
(12) Patent Application Publication
(10) Pub. No.: US 2025/0289784 A1
(43) Pub. Date: Sep. 18, 2025

(54) DIROXIMEL FUMARATE SYNTHESIS

(71) Applicant: BIOGEN MA INC., Cambridge, MA (US)

(72) Inventors: Daw-long Albert Kwok, Gillette, NJ (US); Chao Fei, Waltham, MA (US)

(21) Appl. No.: 18/860,331

(22) PCT Filed: Apr. 26, 2023
(86) PCT No.: PCT/US2023/019961

§ 371 Date: Oct. 25, 2024

Related U.S. Application Data
(60) Provisional application No. 63/335,275, filed on Apr. 27, 2022.

(51) Int. Cl. C07D 207/404; B01J 27/138; B01J 31/26

(52) U.S. Cl. CPC C07D 207/404; B01J 27/138; B01J 31/26; B01J 2231/48; B01J 2531/22

(57) ABSTRACT

Disclosed is a one-pot process (without the isolation of three intermediates) of preparing diroximel fumarate represented by the following structural formula: The method comprises reacting ethylene carbonate with succinimide 5 to form 2-hydroxyethyl succinimide; reacting 2-hydroxyethyl succinimide with maleic anhydride to form a (Z)-4-(2-(2,5-dioxopyrrolidin-1-yl)ethoxy)-4-oxobut-2-enoic acid intermediate, isomerizing the (Z)-4-(2-(2,5-dioxopyrrolidin-1-yl)ethoxy)-4-oxobut-2-enoic acid intermediate to an (E)-4-(2-(2,5-dioxopyrrolidin-1-yl)ethoxy)-4-oxobut-2-enoic acid intermediate, and reacting the (E)-4-(2-10(2,5-dioxopyrrolidin-1-yl)ethoxy)-4-oxobut-2-enoic acid intermediate with methanol to form the product compound.

### RELATED APPLICATION

This application claims the benefit of the filing date, under 35 U.S.C. § 119(e), of U.S. Provisional Application No. 63/335,275, filed on Apr. 27, 2022, the entire contents of which are incorporated herein by reference.

### FIELD OF THE INVENTION

Disclosed is an improved method for preparing diroximel fumarate. The method comprises reacting ethylene carbonate with succinimide to form a 2-hydroxyethyl succinimide intermediate; reacting the 2-hydroxyethyl succinimide intermediate with maleic anhydride in the presence of a catalytic amount of a Lewis acid to form (E)-4-(2-(2,5-dioxopyrrolidin-1-yl)ethoxy)-4-oxobut-2-enoic acid intermediate, and reacting the (E)-4-(2-(2,5-dioxopyrrolidin-1-yl)ethoxy)-4-oxobut-2-enoic acid intermediate with methanol in the presence of a carboxylic acid coupling agent and an acyl transfer catalyst to form diroximel fumarate.

### BACKGROUND OF THE INVENTION

Diroximel fumarate is sold under the brand name Vumerity and is a medication used for the treatment of relapsing forms of multiple sclerosis. Diroximel fumarate was first disclosed in U.S. Pat. No. 8,669,281 and approved for medical use in the United States in October 2019.

Successful development of a new drug requires a cost efficient, high yield synthesis that is amenable to large scale manufacture. U.S. Pat. No. 8,669,281 discloses preparing diroximel fumarate by reacting monomethyl fumarate with 2-hydroxyethyl succinimide in the presence of the coupling agent 2-(1H-benzotriazole-1-yl)-1,1,3,3-tetramethylaminium tetrafluoroborate (hereinafter “TBTU”) as follows:

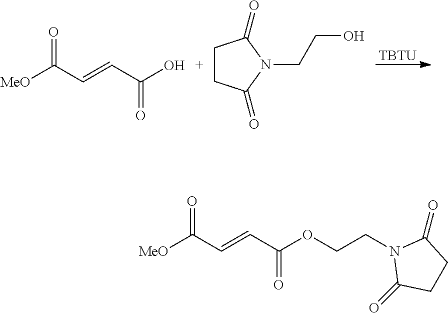

However, U.S. Pat. No. 8,669,281 reports a yield of only 35% for this step.

Commercial manufacture has requirements in addition to high yields for the reactions used in the manufacturing process. The process should use reagents that are safe, inexpensive and compatible with the equipment used in plants. Moreover, the number of process steps are preferably minimized to reduce waste and the need to clean equipment. As such, improved methodology for preparing diroximel fumarate is needed.

### SUMMARY OF THE INVENTION

Disclosed herein is a highly efficient synthesis of diroximel fumarate comprising four reaction steps. The process is shown schematically below:

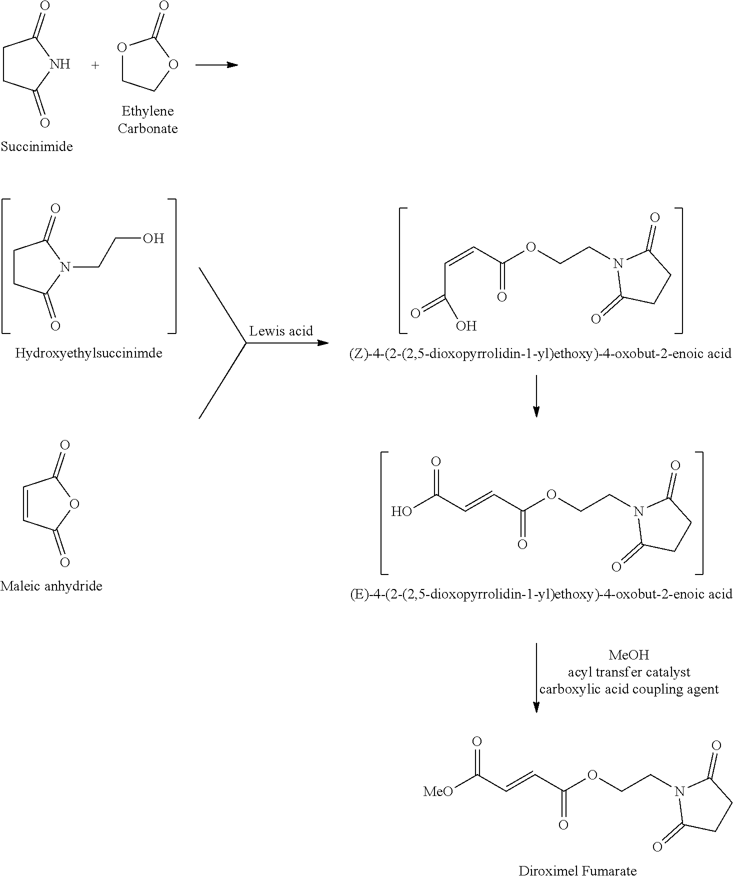

The process is highly efficient and can achieve overall 76% yield when carried out on a pilot plant scale (see Example 3). Additionally, the disclosed process is environmentally friendly. Specifically, the reactions used in the disclosed process can be carried out in “one pot” without isolation of the three intermediates, which efficiently utilizes manufacturing equipment and minimizes solvent usage and waste disposal. In addition, production time is reduced to one third of the production time required for currently used processes. This translates into efficient energy savings. As such, the disclosed process is a highly efficient, cost effective and environmentally compatible process which is ideally suited to commercial production.

In one embodiment, the invention is directed to improved processes for preparing a product compound (III):

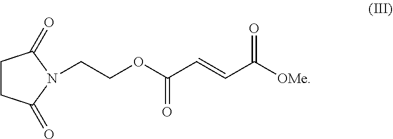 [SMILES: COC(=O)/C=C/C(=O)OCCN1C(=O)CCC1=O]

The process includes the steps of: a) reacting ethylene carbonate with succinimide to form a 2-hydroxyethyl succinimide intermediate (I):

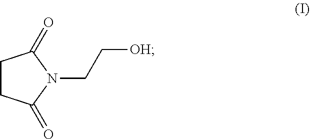

b) reacting the 2-hydroxyethyl succinimide intermediate (I) with maleic anhydride in the presence of a catalytic amount of a Lewis acid to form an (E)-4-(2-(2,5-dioxopyrrolidin-1-yl)ethoxy)-4-oxobut-2-enoic acid intermediate (II):

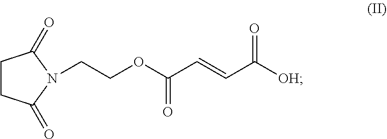 [SMILES: O=C(O)/C=C/C(=O)OCCN1C(=O)CCC1=O]

and
c) reacting the (E)-4-(2-(2,5-dioxopyrrolidin-1-yl)ethoxy)-4-oxobut-2-enoic acid intermediate of formula (II) with methanol in the presence of a carboxylic acid coupling agent and an acyl transfer catalyst to form the product compound (III).

Another embodiment of the invention is a method of preparing a product compound (II) by reacting starting material (I) with maleic anhydride in the presence of a catalytic amount of magnesium bromide (MgBr₂), magnesium chloride (MgCl₂), or magnesium bromide ethyl etherate (MgBr₂.OEt₂) to form product (II).

### DETAILED DESCRIPTION OF THE INVENTION

The disclosed preparation of diroximel fumarate involves four reactions including the in situ isomerization of (Z)-4-(2-(2,5-dioxopyrrolidin-1-yl)ethoxy)-4-oxobut-2-enoic acid to (E)-4-(2-(2,5-dioxopyrrolidin-1-yl)ethoxy)-4-oxobut-2-enoic acid. Advantageously, the reactions can all be carried out in “one pot”, i.e., in one reactor without isolating the intermediate reaction products.

The reaction in step a) between succinimide and ethylene carbonate is in one aspect carried out in the presence of a an amine base. Suitable amine bases are those that do not otherwise interfere with the reaction or cause side reactions. In one aspect, a catalytic amount of the base is used. In one aspect, the amine base is diazabicyclo[5.4.0]undec-7-ene (DBU), 1,5-diazozbicyclo(4.3.0)non-5-ene (DBN), dimethylaminopyridine (DMAP), or 1-methylimidazole. In another aspect, the amine base is 1-methylimidazole 1,8-diazabicycloundec-7-ene (DBU).

The reaction of step a) is carried out at room temperature or elevated temperatures. In one aspect, the reaction of step a) is carried out at elevated temperatures, e.g., temperatures at which the reaction solvent (if used) evaporates during the course of the reaction, e.g., between 50° C.-120° C. In another aspect, step a) is carried out at a temperature between 80° C. and 120° C., 85° C. and 115° C., or 90° C. and 110° C.

In one aspect, a first solvent is added to the reaction in step a) between succinimide and ethylene carbonate. The amount of the first solvent added is sufficient to disperse the reaction mixture in order to facilitate agitation or stirring of the reaction mixture. Suitable solvents include an ethereal solvent, a halogenated solvent, a protic solvent or a polar aprotic solvent such as acetonitrile or dipolar aprotic solvent such dimethyl formamide or dimethyl sulfoxide. The solvent is selected so that the solvent has a boiling point that is lower than the final reaction temperature, so that the solvent distills as the reaction progresses (e.g., when the boiling point of the solvent is about 10° C. to 60° C. lower than the final reaction temperature). This allows for a minimal amount of solvent to be used. In an aspect, the first solvent has a boiling point that is between 40° C. and 100° C., or between 50° C. and 90° C. Examples of suitable solvent include acetonitrile, acetone, tetrahydrofuran, 2-methyl tetrahydrofuran, methyl acetate, ethyl acetate, isopropyl acetate, methanol, ethanol, isopropanol or a mixture thereof. In another aspect, the first solvent is acetonitrile.

The reaction in step b) between 2-hydroxyethyl succinimide and maleic anhydride is carried out in the presence of a catalytic amount of a Lewis acid. Suitable Lewis acids include magnesium bromide (MgBr₂), magnesium bromide ethyl etherate (MgBr₂.OEt₂), magnesium chloride (MgCl₂), magnesium iodide (MgI₂), lithium chloride (LiCl), lithium bromide (LiBr) and nickel chloride (NiCl₂). MgBr₂ and MgBr₂.OEt₂ gave superior yields (>96%) compared with other Lewis acids tested. (See Table 1 in Example 4). Exemplary catalytic amounts include 0.01 to 0.20 or 0.05 to 0.15 molar equivalents based on moles of ethylene carbonate. Magnesium chloride (MgCl₂) gave similar results when the reaction time was extended.

The reaction in step b) may be carried out in a second solvent. The second solvent is added in step b) prior to the addition of the Lewis acid. Suitable second solvents include acetone, 2-butanone, 2-pentanone, 3-pentanone, tetrahydrofuran, 2-methyl tetrahydrofuran, acetonitrile, ethyl acetate, isopropyl acetate, and mixtures thereof. In some examples, the second solvent is 2-butanone. In other examples, the second solvent is a mixture of acetonitrile and 2-butanone. In some examples, the second solvent is a 2:1 v/v mixture of 2-butanone and acetonitrile.

The Lewis acid may be added to the reactor neat, i.e., as a solid. However, it is difficult to control the rate of addition of a solid during scale-up processes. Further, it was found that crusting in the reactor occurs with the addition of anhydrous magnesium bromide or magnesium bromide etherate, even at laboratory scale (e.g., 50 g). “Crusting” refers to the formation of a precipitate on reactor surface, generally on the reactor wall at or slightly above the fill level of the reaction mixture. Crusting can be reduced by adding the Lewis acid as a solution, i.e., wherein the Lewis acid is dissolved in a charging solvent, for example a ketone, e.g. butanone or pentanone. For a controlled rate of addition, the solution containing the dissolved Lewis acid is added via pump.

Crusting is further reduced when the Lewis acid is added a solution in which the Lewis acid is dissolved in butanone as the charging solvent. It was found that by charging magnesium bromide in 1 volume of butanone relative to the solution of HES in a mixture of 2 volume of butanone and 1 volume of acetonitrile the crusting is minimized or eliminated. In some examples, the Lewis acid of step b) is added to the reaction in step b) in a solution comprising butanone. In some examples, the reaction solution of step b) is a 3:1 v/v mixture of butanone/acetonitrile butanone and acetonitrile after the addition of the Lewis acid. However, other amounts of ketone may be used so that the volume ratio (v/v) of butanone to acetonitrile in the reaction mixture after the charging of the Lewis acid may vary, i.e., the reaction mixture may comprise a 5:1, a 4:1, a 3:2, a 3:1 or a 1:1 v/v mixture of butanone and acetonitrile, or may be butanone only.

In one aspect, a second solvent is added to the reaction in step b) between hydroxyethyl succinimide and maleic anhydride. Exemplary solvents are selected from acetone, 2-butanone, 2-pentanone, 3-pentanone, tetrahydrofuran, 2-methyl tetrahydrofuran, acetonitrile, ethyl acetate, isopropyl acetate and a mixture thereof. In another aspect, the second solvent is 2-butanone or a mixture of butanone and acetonitrile.

In one aspect, the reaction of step b) is carried out at a temperature between 60° C. and 100° C. Alternatively, the reaction of step b) is carried out at a temperature between 70° C. and 90° C., between 75° C. and 85° C., or at 80° C.

The reaction in step c) between (E)-4-(2-(2,5-dioxopyrrolidin-1-yl)ethoxy)-4-oxobut-2-enoic acid and methanol is carried out in the presence of a carboxylic coupling acid agent and an acyl transfer catalyst.

A “carboxylic acid coupling reagent” activates the hydroxyl group of a carboxylic acid towards nucleophilic substitution by, for example, an alcohol, such as the alcohol group of methanol. Carboxylic acid coupling reagents are known in the art and include, e.g., carbodiimides, phosphonium reagents, aminium/uranium-imonium reagents, N-ethoxycarbonyl-2-ethoxy-1,2-dihydroquinoline, 2-propanephosphonic acid anhydride, 4-(4,6-dimethoxy-1,3,5-triazin-2-yl)-4-methylmorpholinium salt, bis-trichloromethylcarbonate, 1,1′-carbonyldiimidazole, mesyl chloride, propylphosphonic anhydride, pivaloyl chloride, oxalyl chloride and thionyl chloride. In one aspect, the carboxylic coupling acid agent of step c) is selected from a carbodiimide, an aminium/uranium-imonium reagent, N-ethoxycarbonyl-2-ethoxy-1,2-dihydroquinoline, 2-propanephosphonic acid anhydride, 4-(4,6-dimethoxy-1,3,5-triazin-2-yl)-4-methylmorpholinium salt, bis-trichloromethylcarbonate, and 1,1′-carbonyldiimidazole. In another aspect, the carboxylic acid coupling agent is a carbodiimide. In yet another aspect, the carbodiimide is N-(3-dimethylaminopropyl)-N′-ethylcarbodiimide or a salt thereof. In another aspect still, the carbodiimide is N-(3-dimethylaminopropyl)-N′-ethylcarbodiimide hydrochloride (EDC.HCl).

The reaction between a carbodiimide and a carboxylic acid is believed to result in an O-acylisourea intermediate. An acyl transfer catalyst is commonly added to carbodiimide couplings to further accelerate the formation of the ester and suppress acyl urea impurities. The acyl transfer catalyst is a better nucleophile than an alcohol and forms an activated intermediate. This activated intermediate readily reacts with alcohols to form the product ester. The mediation of carbodiimide couplings with an acyl transfer catalyst is described in Chan and Cox, J. Org. Chem., 72:8863 (2007). Examples of suitable acyl transfer catalysts for carbodiimide couplings of step 3) are selected from dimethylaminopyridine, 1-methylimidazole, and 1H-benzo[d][1,2,3]triazol-1-ol (HOBt). In one aspect, the acyl transfer catalyst for carbodiimide couplings is N-methylimidazole (NMI).

The final product compound represented by structural formula (III) is isolated following the reaction of step c). In one aspect, the compound of structural formula (III) is isolated by crystallization, e.g., by addition of an antisolvent such as isopropanol followed by cooling to a low temperature and then followed by filtration.

The invention is illustrated by the following examples, which are not intended to be limiting in any way.

### EXEMPLIFICATION

### Glossary of Terms

ACN acetonitrile
DBU 1-methylimidazole 1,8-diazabicycloundec-7-ene
g gram
h hour
HES hydroxyethyl succinimide
HPLC High Performance Liquid Chromatography
L liter
m mole
min minute
mL milliliter
mm millimeter
mol. eq. molar equivalents based on moles of ethylene carbonate
NMI 1-methylimidazole
μl microliter
nm nanometers
TFA triflouroacetic acid
V. volume equivalents
% (v/v) volume percentage

### Example 1—Preparation of Diroximel Fumarate with MgBr₂.OEt₂

A reactor was charged with ethylene carbonate (50.0 g, 0.568 m, 1.00 mol. eq.). Acetonitrile (50 mL, 1.0 V.) was added to facilitate stirring. Succinimide (59.6 g, 0.602 m, 1.06 mol. eq.) was added to the reactor, followed by the addition of DBU (1.74 g, 0.0114 m, 0.02 mol. eq.). The reactor was then heated to 100° C. and the reaction was monitored for the depletion of ethylene carbonate, which took about six hours. During the reaction, acetonitrile was removed from the reactor by distillation. Next, the reactor was cooled to 70° C. and 2-butanone (200 mL, 4.0 V) was added. Maleic anhydride (72.4 g, 0.738 m, 1.3 mol. eq.) was added to the reactor, followed by acetic acid (4.09 g, 0.0682 m, 0.12 mol. eq.) Next, magnesium bromide ethyl etherate (MgBr₂.OEt₂) (11.0 g, 0.0426 m, 0.075 mol. eq.) was added to the reactor over 1 h with agitation to minimize the aggregation of solids in the reactor. The reactor was then heated to 80° C. and the reaction was monitored by HPLC for the depletion of 2-hydroxyethyl succinimide (HES), which took about 24 hours. The reactor was cooled to 60° C. over a two hour period, and then methanol (36.4 g, 1.136 m, 2.0 mol. eq.) was added to the reactor. Next, the reactor was further cooled to 35° C. over a two-hour period. N-methylimidazole (NMI) (0.699 g, 0.00852 m, 0.015 mol. eq.) was added to the reactor, followed by the addition of N-(3-dimethylaminopropyl)-N′-ethylcarbodiimide hydrochloride (EDC.HCl) (163.3 g, 0.852 m, 1.5 mol. eq.) over a 1-hour period. The reaction was monitored by HPLC for the depletion of the 4-(2-(2,5-dioxopyrrolidin-1-yl)ethoxy)-4-oxobut-2-enoic acid intermediate, which took about 3 hours. With the reactor temperature maintained at 35° C., water (25.0 ml, 0.5 V) was added to the reactor, followed by isopropanol (400 mL, 8.0 V). The reactor was heated to 55° C. to make a homogeneous solution, and then the reactor was cooled to 0° C. over 3 hours and then stirred at 0° C. for 1 hour. The reactor contents were collected by filtration and then the filter cake was washed with 250 mL (5.0 V) of pre-cooled (0° C.) acetone/water mixture (1:4 v/v). The cake was then dried under house vacuum (with nitrogen bleed) at 50° C. for 24 h to afford 107.1 g (73.9% molar yield) of white title compound.

### Example 2—Preparation of Diroximel Fumarate with MgBr₂ and Butanone/Acetonitrile Mixture

A reactor was charged with ethylene carbonate (9.20 g, 0.104 m, 1.00 mol. eq.). Acetonitrile (9.2 mL, 1.0 V.) was added to facilitate stirring. Succinimide (10.97 g, 0.111 m, 1.06 mol. eq.) was added to the reactor, followed by DBU (0.316 g, 0.00208 m, 0.02 mol. eq.). The reactor was then heated to 100° C. and the reaction was monitored by GC for the depletion of ethylene carbonate, which took about six hours. During the reaction, acetonitrile was removed from the reactor by distillation. Next, the reactor was cooled to 70° C. and 2-butanone (18.4 mL, 2.0 V) and acetonitrile (9.2 mL, 1.0 V) were added. Maleic anhydride (12.75 g, 0.130 m, 1.25 mol. eq.) was added to the reactor. Next, a solution of magnesium bromide (MgBr₂) (1.44 g, 0.0078 m, 0.075 mol. eq.) in 2-butanone (9.2 mL, 1.0 V) was added to the reactor over a 1-hour period. The reactor was then heated to 80° C. and the reaction was monitored by HPLC for the depletion of 2-hydroxyethyl succinimide (HES), which took about 24 hours. The reactor was then cooled to 60° C. over a 2-hour period, and then methanol (6.66 g, 0.208 m, 2.0 mol. eq.) was added to the reactor, and then the reactor was further cooled to 35° C. over a 2-hour period. N-methylimidazole (NMI) (0.128 g, 0.00156 m, 0.015 mol. eq.) was added to the reactor, followed by the addition of N-(3-dimethylaminopropyl)-N′-ethylcarbodiimide hydrochloride (EDC.HCl) (31.90 g, 0.166 m, 1.60 mol. eq.) over a 1-hour period. The reaction was monitored for the depletion of the 4-(2-(2,5-dioxopyrrolidin-1-yl)ethoxy)-4-oxobut-2-enoic acid intermediate, which took about 3 hours. With the reactor temperature maintained at 35° C., water 1.84 mL, 0.2 V) was added to the reactor, followed by isopropanol (73.6 mL, 8.0 V). The reactor was heated to 55° C. to make a homogeneous solution; then the reactor was cooled to 0° C. over 3 hours; and then stirred at 0° C. for 1 hour. The reactor contents were collected by filtration and then the filter cake was washed with 46 mL (5.0 V) of pre-cooled (5° C.) acetone/water mixture (1:4 v/v). The cake was then dried under house vacuum (with nitrogen bleed) at 50° C. for 24 h to afford 19.89 g (74.9% molar yield) of white title compound.

### Example 3—Preparation of Diroximel Fumarate 50 Kilogram Scale

A reactor was charged with acetonitrile (3.4 kg, 0.17 V) and ethylene carbonate (25.0 kg, 1.00 mol. eq.) was added. Additional acetonitrile (2 kg, 0.1 V) was added to facilitate stirring. Succinimide (29.9 kg, 1.06 mol. eq.) was added to the reactor, followed by acetonitrile (3.2 kg, 0.16 V). The reactor was brought to a temperature of 75-85° C. within a 2.5-hour period and then DBU (0.20 g, 0.005 mol. eq.) was added. The reactor was then heated to 90-100° C. within 1 hour and then held at that temperature for an additional 1 hour. Additional DBU (0.65 kg, 0.015 mol. eq.) was added to the reactor within a 3-hour period as the reaction temperature was maintained at 90-110° C. The reactor was then heated to 100-110° C. within 1 hour and then held at that temperature for 5 hours. During the reaction, acetonitrile was evaporated and collected by a condenser. Next, the reactor was cooled to 45-55° C. within a 2.5-hour period and the reaction was monitored by GC until less than 0.5% ethylene carbonate remained. 2-Butanone (41 kg, 2.0 V) was added to the reactor, followed by acetonitrile (19 kg, 0.97 V) and then maleic anhydride (35 kg, 1.26 mol. eq.)

To a charging device was added 2-Butanone (24 kg, 1.2 V) followed by the slow addition of magnesium bromide (MgBr₂) (4 kg, 0.077 mol. eq.) with agitation while keeping the charging device temperature below 55° C. The resulting MgBr₂/2-Butanone solution was added to the reactor over a 1.5-h period. The charging device was rinsed with 2-Butanone (10 kg) and the MEK was added to the reactor.

The reactor was then heated to 80-90° C. over a 2-hour period and then held at that temperature for 30 min, and the reaction was monitored for the depletion of 2-hydroxyethyl succinimide (HES) until less than 0.4% remained and for the depletion of maleic anhydride until less than 1.2% remained. The reactor was then cooled to 55-65° C. over a 1.5-hour period, and then methanol (18.4 kg, 2.0 mol. eq.) was added to the reactor, and then the reactor was further cooled to 30-40° C. over a 1-hour period. N-methylimidazole (NMI) (0.35 kg, 0.015 mol. eq.) was added to the reactor, followed by the addition of N-(3-dimethylaminopropyl)-N′-ethylcarbodiimide hydrochloride (EDC.HCl) (90 kg, 1.66 mol. eq.) within a 4.4-hour period. The reaction was monitored for the depletion of the cis-4-(2-(2,5-dioxopyrrolidin-1-yl)ethoxy)-4-oxobut-2-enoic acid intermediate, which took about 4 hours.

Water (5 kg, 0.2 V) was added to the reactor, followed by isopropanol (156 g, 7.9 V). The reactor was heated to 55-65° C. over a 1-hour period to make a homogeneous solution; then the reactor was cooled to-5° C. over a 3-hour period; and then stirred at −5° C. for 3 hour. The reactor contents were collected by filtration and then the filter cake was washed with 100 L of pre-cooled (5° C.) acetone/water mixture (1:4 v/v). The cake was then dried at 45-55° C. for 24 hours to afford 55.3 kg (99.2% purity, 75.7% molar yield) of the white title compound.

### Example 4—Comparison of Lewis Acid Catalysts

The reaction between 2-hydroxyethyl succinimide and maleic anhydride is carried out in the presence of a catalytic amount of a Lewis acid. The results of a comparative study of various Lewis acids is shown in Table 1:

TABLE 1
Comparison of Lewis Acid Catalysts

| | | Conversion (%) at | Conversion (%) at |
|---|---|---|---|
| Catalyst | Loading (eq.) | 20-24 h | 36 h |
| LiCl | 0.1 | 1.0 | — |
| LiBr | 0.1 | 1.4 | — |
| MgCl₂ | 0.1 | 69.2 | 96% |
| MgBr₂ | 0.1 | 96.5 | — |
| MgBr₂•OEt₂ | 0.1 | 96.2 | — |
| MgI₂ | 0.1 | 42.9 | — |
| NiCl₂ | 0.1 | 15.9 | — |

Magnesium bromide (MgBr₂) and magnesium bromide etherate (MgBr₂.OEt₂) provided much higher yields (>96%) compared to the other Lewis acids tested for a reaction time lasting 20-24 h. However, magnesium chloride (MgCl₂) gives similar yields when the reaction time is extended

What is claimed is:

1. A method of preparing a product compound represented by the following structural formula (III): 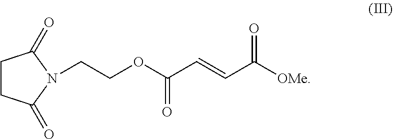 [SMILES: COC(=O)/C=C/C(=O)OCCN1C(=O)CCC1=O]
comprising the steps of:
a) reacting ethylene carbonate with succinimide to form a 2-hydroxyethyl succinimide intermediate represented by the following structural formula (I): 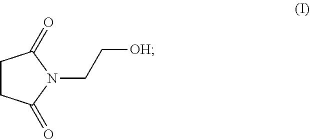
b) reacting the hydroxyethyl succinimide intermediate of structural formula (I) with maleic anhydride in the presence of a catalytic amount of a Lewis acid to form an (E)-4-(2-(2,5-dioxopyrrolidin-1-yl)ethoxy)-4-oxobut-2-enoic acid intermediate represented by the following structural formula (II): 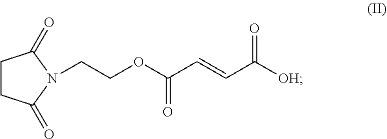 [SMILES: O=C(O)/C=C/C(=O)OCCN1C(=O)CCC1=O]
and
c) reacting the (E)-4-(2-(2,5-dioxopyrrolidin-1-yl)ethoxy)-4-oxobut-2-enoic acid intermediate of formula (II) with methanol in the presence of a carboxylic acid coupling agent and an acyl transfer catalyst to form the product compound (III).

2. The method of claim 1, wherein steps a), b), and c) are carried out in one pot without the isolation of intermediates.

3. The method of claim 1 or claim 2, wherein step a) is conducted in the presence of an amine base.

4. The method of claim 3, wherein the amine base is diazabicyclo[5.4.0]undec-7-ene (DBU), 1,5-diazozbicyclo(4.3.0)non-5-ene (DBN), dimethylaminopyridine (DMAP), or 1-methylimidazole.

5. The method of claim 3 or claim 4, wherein the amine base is diazabicyclo[5.4.0]undec-7-ene (DBU).

6. The method of any one of claims 1-5, wherein the reaction in step a) is carried out at a temperature between 50° C. and 120° C.

7. The method of claim 6, wherein the reaction in step a) is carried out at a temperature between 80° C. and 120° C., 85° C. and 115° C., or 90° C. and 110° C.

8. The method of any one of claims 1-7, wherein a first solvent is added in step a).

9. The method of claim 8, wherein the first solvent has a boiling point that is between 50° C. and 90° C.

10. The method of any one of claims 7-9, wherein the first solvent is selected from acetonitrile, acetone, tetrahydrofuran, 2-methyl tetrahydrofuran, methyl acetate, ethyl acetate, isopropyl acetate, methanol, ethanol, isopropanol, and mixtures thereof.

11. The method of claim 10, wherein the first solvent is acetonitrile.

12. The method of any one of claims 1-11, wherein a second solvent is added in step b) prior to the addition of the Lewis acid.

13. The method of claim 12, wherein the second solvent is selected from acetone, 2-butanone, 2-pentanone, 3-pentanone, tetrahydrofuran, 2-methyl tetrahydrofuran, acetonitrile, ethyl acetate, isopropyl acetate, and mixtures thereof.

14. The method of claim 12 or claim 13, wherein the second solvent is 2-butanone or a mixture of acetonitrile and 2-butanone.

15. The method of any one of claims 12-14, wherein the second solvent is a 2:1 v/v mixture of 2 butanone and acetonitrile.

16. The method of any one of claims 1-15, wherein the Lewis acid of step b) is magnesium chloride (MgCl₂), magnesium bromide (MgBr₂), or magnesium bromide ethyl etherate (MgBr₂.OEt₂).

17. The method of any one of claims 1-16, wherein the Lewis acid of step b) is added to the reaction in step b) in a solution comprising a charging solvent.

18. The method of claim 17, wherein the charging solvent is selected from acetone, 2-butanone, 2-pentanone, 3-pentanone, tetrahydrofuran, 2-methyl tetrahydrofuran, acetonitrile, ethyl acetate, isopropyl acetate, and mixtures thereof.

19. The method of claim 17 or claim 18, wherein the charging solvent is butanone.

20. The method of any one of claims 17-19, wherein step b) is carried out in a 3:1 v/v mixture of butanone and acetonitrile after the addition of the Lewis acid.

21. The method of any one of claims 1-20, wherein the reaction of step b) is carried out at a temperature between 60° C. and 100° C., for example at 80° C.

22. The method of any one of claims 1-20, wherein the carboxylic acid coupling agent of step c) is selected from a carbodiimide, an aminium/uranium-imonium reagent, N-ethoxycarbonyl-2-ethoxy-1,2-dihydroquinoline, 2-propanephosphonic acid anhydride, 4-(4,6-dimethoxy-1,3,5-triazin-2-yl)-4-methylmorpholinium salt, bis-trichloromethylcarbonate, and 1,1′-carbonyldiimidazole.

23. The method of claim 22, wherein the carboxylic acid coupling agent is a carbodiimide.

24. The method of claim 23, wherein the carbodiimide is N-(3-dimethylaminopropyl)-N′-ethylcarbodiimide or a salt thereof.

25. The method of claim 24, wherein the carbodiimide is N-(3-dimethylaminopropyl)-N′-ethylcarbodiimide hydrochloride.

26. The method of any one of claims 1-25, wherein the acyl transfer catalyst of step 3) is selected from dimethylaminopyridine, 1-methylimidazole, and 1H-benzo[d][1,2,3]triazol-1-ol (HOBt).

27. The method of any one of claims 1-26, wherein the method further comprises the step of: d) isolating the product compound represented by structural formula (III).

28. The method of claim 27, wherein the product compound is isolated by crystallization.

29. A method of preparing a product compound represented by the following structural formula (II): 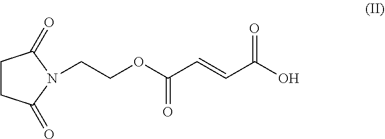 [SMILES: O=C(O)/C=C/C(=O)OCCN1C(=O)CCC1=O]
by reacting a starting material having the following structural formula (I): 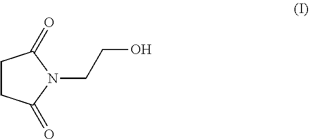
with maleic anhydride in the presence of a catalytic amount of MgCl₂, MgBr₂ or MgBr₂.OEt₂ to form the product represented by structural formula (II).

30. The method of claim 29, wherein the reaction is carried out in acetonitrile, 2-butanone, 3-pentanone, 2-pentanone, or a mixture thereof.

31. The method of claim 29 or claim 30, wherein the reaction is carried out in a solution comprising 2-butanone and acetonitrile.

32. The method of any one of claims 29-31 wherein MgBr₂ is added to the reaction as a solution comprising butanone.

33. The method of claim 31 or 32, wherein the reaction is carried out in a 3:1 v/v mixture of butanone and acetonitrile.
